(19) United States
(12) Patent Application Publication
(10) Pub. No.: US 2023/0094702 A1
(43) Pub. Date: Mar. 30, 2023

(54) Inner Plug Constituting Nozzle And Eye Drop Container

(71) Applicant: Fujimori Kogyo Co., Ltd., Tokyo (JP)

(72) Inventors: Yoshitoh Tanaka, Yokohama-shi (JP); Asako Kanazawa, Yokohama-shi (JP); Toyoaki Suzuki, Yokohama-shi (JP)

(21) Appl. No.: 17/802,077

(22) PCT Filed: Feb. 26, 2021
(86) PCT No.: PCT/JP2021/007433

§ 371 Date: Aug. 24, 2022

(30) Foreign Application Priority Data
Feb. 28, 2020 (JP) 2020-033261

(51) Int. Cl. B65D 47/18; A61J 1/14; A61F 9/00

(52) U.S. Cl. CPC B65D 47/18; A61F 9/0008; A61J 1/1475

(57) ABSTRACT

The inner plug is mounted in an opening part of a container body and constitutes a nozzle part. The inner plug is constituted of a resin composite including a cyclic olefin copolymer and a polyethylene resin. A proportion of the cyclic olefin copolymer in 100 parts by weight of the resin composite is 25 to 85 parts by weight, a proportion of the polyethylene resin therein is 15 to 75 parts by weight, and a total amount of the cyclic olefin copolymer and the polyethylene resin is 80 to 100 parts by weight.

### FIELD OF THE INVENTION

The present invention relates to an inner plug constituting a nozzle and an eye drop container including the same.

Priority is claimed to Japanese Patent Application No. 2020-033261, filed Feb. 28, 2020, the content of which is incorporated herein by reference in its entirety.

### BACKGROUND OF THE INVENTION

In the related art, regarding eye drop containers for storing eye drops, resin containers made of polyethylene (PE), polypropylene (PP), polyethylene terephthalate (PET), or the like are widely used. Japanese Unexamined Patent Application, First Publication No. 2015-107322 describes that an eye drop container including a container body constituted of a resin molded article is seal-packaged with a predetermined laminated film. The container body includes a storage part for eye drops and a nozzle part protruding from the storage part and is constituted such that eye drops can be applied from the eye drop container by pressurizing the storage part with the fingers while pointing the nozzle part toward the eye.

Regarding a resin for constituting an eye drop container in the related art, polyethylene or the like is inexpensive but has a disadvantage that chemical agents (active components or the like) are likely to be adsorbed thereby. Regarding a packaging container such as a packaging bag, a cyclic olefin copolymer is known as a resin having excellent non-adsorption properties with respect to content components (for example, refer to PCT International Publication No. WO 2003/043895 and PCT International Publication No. WO 2004/080370).

### SUMMARY OF THE INVENTION

### Technical Problem

In order to suppress sorption (adsorption or absorption) of eye drops in a storage part, there is a need to employ a material which is unlikely to sorb active components of eye drops. However, according to the investigation of the inventors, it has been discovered that when a cyclic olefin copolymer is used in an inner plug, there is a problem due to the cyclic olefin copolymer harder than a resin such as PE, PP, or PET described above.

The present invention has been made in consideration of the foregoing circumstances and aims to provide an inner plug, in which sorption (adsorption, absorption) of eye drops can be suppressed, and an eye drop container including the same.

### Solution to Problem

In order to resolve the foregoing problems, according to an aspect of the present invention, there is provided an inner plug configured to be mounted in an opening part of a container body and constituting a nozzle. The inner plug is constituted of a resin composite including a cyclic olefin copolymer and a polyethylene resin. A proportion of the cyclic olefin copolymer in 100 parts by weight of the resin composite is 25 to 85 parts by weight, a proportion of the polyethylene resin therein is 15 to 75 parts by weight, and a total amount of the cyclic olefin copolymer and the polyethylene resin is 80 to 100 parts by weight.

The inner plug may have a flange part which comes into contact with a tip surface of the opening part, and a leg part which comes into contact with an inner surface of the opening part.

The nozzle may have a nozzle hole penetrating the inner plug.

The resin composite may contain a rubber or an elastomer.

The resin composite may include at least one kind of a copolymer of cyclic olefins different from each other (COP) and a copolymer of a cyclic olefin and an acyclic olefin (COC).

According to another aspect of the present invention, there is provided an eye drop container including the inner plug, and a container body that has an opening part in which the inner plug is mounted.

The container body may have a layer including a cyclic olefin copolymer on at least a surface in contact with a liquid content.

### Advantageous Effects of Invention

The inner plug according to the aspect of the present invention has a liquid contact part that is constituted of a layer including a cyclic olefin copolymer having excellent non-sorption properties, and a cushioning part that is provided in an outer circumferential part in contact with an opening part. Therefore, sorption (adsorption, absorption) of eye drops can be suppressed.

In addition, according to the eye drop container of another aspect, eye drops can be stably stored while sorption (adsorption, absorption) of eye drops is suppressed.

### BRIEF DESCRIPTION OF THE DRAWINGS

FIG. 1 is a cross-sectional view illustrating an example of the present invention, in which an inner plug of an embodiment is mounted in an opening part of a container body.

### DETAILED DESCRIPTION OF THE INVENTION

Hereinafter, the present invention will be described with reference to the drawings on the basis of a preferred embodiment.

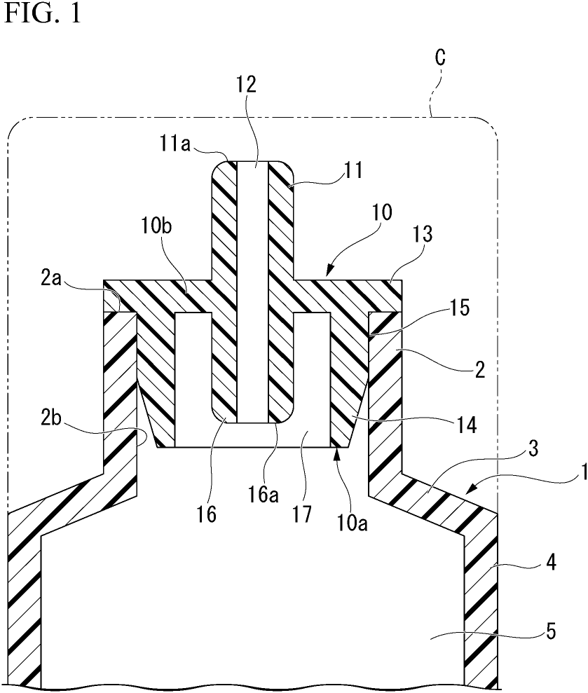

FIG. 1 illustrates an example in which an inner plug 10 of an embodiment is mounted in an opening part 2 of a container body 1. An eye drop container of the embodiment is constituted of a dripping container including the inner plug 10 that has a nozzle part 11, and the container body 1 that has the opening part 2 in which the inner plug 10 is mounted. The container body 1 has a storage part 5 for storing a content in a direction opposite to a direction in which the nozzle part 11 protrudes from the opening part 2. The container body 1 of this embodiment has a bottomed cylindrical shape, the opening part 2 has a cylindrical shape having a smaller outer diameter than the container body 1, the nozzle part 11 also has a cylindrical shape, and all of these are coaxially disposed. A content is a content constituted of a liquid or a content including a liquid.

When the inner plug 10 is mounted in the opening part 2, the nozzle part 11 of the inner plug 10 constitutes a nozzle of the container body 1. In FIG. 1, in a general stationary state of the eye drop container, the nozzle part 11 is disposed above, and the storage part 5 is disposed below. When the eye drop container is used, the nozzle part 11 is disposed downward or obliquely downward. In addition, when the eye drop container is stored, the nozzle part 11 may be placed sideways.

The nozzle part 11 has a nozzle hole 12 leading to the storage part 5. A content stored in the storage part 5 is dripped to the outside of the container body 1 through the nozzle hole 12. The inner plug 10 of the embodiment has a disk-shaped flange part 13 which comes into contact with an annular tip surface 2a of the opening part 2, a cylindrical leg part 14 which has a narrowed lower end coming into contact with an inner surface 2b of the opening part 2 in an outer circumferential surface 15, and a cylindrical inner cylinder part 16 which has a shape in which the nozzle part 11 extends toward the storage part 5 on an inner side of the leg part 14. The nozzle hole 12 is formed to have a continuously linear shape to a tip 16a of the inner cylinder part 16. An annular clearance part 17 is formed between an inner surface of the leg part 14 and an outer surface of the inner cylinder part 16. In this example, a lower end of the inner cylinder part 16 is positioned above the lower end of the leg part 14. An upper part of the leg part 14 has a cylindrical shape, and a lower part thereof has a tapered shape narrowing downward in a conical shape. All of the foregoing cylindrical constituent elements can also have a polygonal tubular shape as necessary.

Although it is not particularly illustrated, the inner plug 10 can also be constituted such that the inner side of the leg part 14 is filled with a resin up to an inner surface of the nozzle hole 12 so as not to have the inner cylinder part 16 and the clearance part 17 of the embodiment. In this case, even if the leg part 14 has a large wall thickness, defective molding can be suppressed by molding the leg part 14 from a resin composite (which will be described below) including a cyclic olefin copolymer. The nozzle part 11, the flange part 13, and the like can be integrally molded with the leg part 14.

The inner plug 10 is constituted of a resin composite including a cyclic olefin copolymer and a polyethylene resin. Accordingly, a liquid contact part 10a of the inner plug 10 is also constituted of a resin composite including a cyclic olefin copolymer. The liquid contact part 10a of the inner plug 10 is at least a part or the entirety of parts which may come into contact with a liquid (content). Examples of a part which may constitute the liquid contact part 10a include an inner surface of the nozzle part 11 in the nozzle hole 12, an inner surface of the flange part 13, an inner surface and an outer surface of the leg part 14, and an inner surface and an outer surface of the inner cylinder part 16.

Here, the inner surfaces of the nozzle part 11, the leg part 14, and the inner cylinder part 16 are surfaces on an inner side in a radial direction centering on the nozzle hole 12. In addition, the inner surface of the flange part 13 is a surface on a side opposite to a side where the nozzle part 11 protrudes toward a tip 11a. The outer surfaces thereof are surfaces on a side opposite to the respective inner surfaces. Since the inner plug 10 is integrally molded from the foregoing resin composite, all parts other than the liquid contact part 10a are constituted of a resin composite including a cyclic olefin copolymer as an inner plug main body 10b.

The inner plug 10 is molded from a resin composite including a cyclic olefin copolymer as a resin having excellent non-sorption properties (non-adsorption properties). Examples of a cyclic olefin copolymer generally include so-called copolymers of cyclic olefins different from each other (COP), and copolymers of a cyclic olefin and an acyclic olefin (COC). Accordingly, sorption of a content with respect to the inner plug 10 can be suppressed.

Examples of a copolymer of cyclic olefins different from each other (COP) used in the foregoing resin composite include a copolymer of cyclic olefins of two or more kinds, and hydrogenated products thereof. A copolymer of cyclic olefins different from each other (COP) is preferably an amorphous polymer and is more preferably a ring-opened polymer of cyclic olefins from metathesis or the like, or hydrogenated products thereof. A copolymer of cyclic olefins different from each other (COP) has a higher content proportion of alicyclic structures than a copolymer of a cyclic olefin and an acyclic olefin (COC) or the like and has excellent non-sorption properties (non-adsorption properties).

Examples of a copolymer of a cyclic olefin and an acyclic olefin (COC) used in the foregoing resin composite include a copolymer of at least one kind of a cyclic olefin and at least one kind of an acyclic olefin, and hydrogenated products thereof. A copolymer of a cyclic olefin and an acyclic olefin (COC) is preferably an amorphous polymer and is more preferably a copolymer of a cyclic olefin and ethylene, or hydrogenated products thereof.

A cyclic olefin used as a constituent monomer of the foregoing cyclic olefin copolymer is an unsaturated hydrocarbon (olefin) having at least one ring structure. Examples thereof include at least one kind of vinyl cycloalkane having cycloalkane with a number of carbon atoms of 3 to 20 and derivatives thereof, monocycloalkene with a number of carbon atoms of 3 to 20 and derivatives thereof, and a cyclic olefin having a norbornene skeleton (norbornene-based monomer).

Examples of the foregoing norbornene-based monomer include bicyclo[2.2.1]-2-heptene (norbornene) and derivatives thereof. Examples of a norbornene derivative include a compound which has a substituent group such as an alkyl group, a compound which has two or more unsaturated bonds as norbornadiene, and a compound which has three or more ring structures and in which two ring structures thereof constitute a norbornene skeleton. Examples of a norbornene-based monomer having three or more ring structures include tricyclo[5.2.1.0(2, 6)]decene (dihydrodicyclopentadiene), a compound in which one or more molecules of cyclopentadiene are added to norbornene or dihydrodicyclopentadiene by Diels-Alder reaction (for example, tetracyclododecene, pentacyclopentadecene, or hexacycloheptadecene), hydrogenated products thereof, isomers in which positions of double bonds differ, and an alkyl substituent.

Examples of an acyclic olefin used as a constituent monomer of the foregoing copolymer of a cyclic olefin and an acyclic olefin (COC) include α-olefins such as ethylene, propylene, 1-butene, 1-pentene, 1-hexene, 1-heptene, and 1-octene; and alkenes such as 3-decene and 3-dodecene.

It is preferable that resin components of a resin composite constituting the inner plug 10 including the liquid contact part 10a and the inner plug main body 10b be a mixture including at least one kind of a cyclic olefin copolymer and at least one kind of a polyethylene resin. A proportion of the cyclic olefin copolymer in 100 parts by weight of the foregoing resin composite is preferably 25 to 85 parts by weight and is more preferably equal to or higher than 30 parts by weight.

Examples of a polyethylene resin used in the foregoing resin composite include a homopolymer of ethylene (ethylene homopolymer), linear low-density polyethylene in which ethylene and an α-olefin (1-butene or the like) with a number of carbon atoms of 4 are copolymerized (C4-LLDPE), linear low-density polyethylene in which ethylene and an α-olefin (1-hexene or the like) with a number of carbon atoms of 6 are copolymerized (C6-LLDPE), linear low-density polyethylene in which ethylene and an α-olefin (1-octene or the like) with a number of carbon atoms of 8 are copolymerized (C8-LLDPE), an ethylene-vinyl acetate copolymer (EVA), and an ethylene-vinyl alcohol copolymer (EVOH).

A proportion of a polyethylene resin in the foregoing resin composite in 100 parts by weight of the foregoing resin composite is preferably 15 to 75 parts by weight and is more preferably equal to or lower than 70 parts by weight. In the foregoing resin composite, the total amount of the cyclic olefin copolymer and the polyethylene resin is preferably 80 to 100 parts by weight, is more preferably equal to or higher than 85 parts by weight, is further preferably equal to or higher than 90 parts by weight, and is particularly preferably equal to or higher than 95 parts by weight.

A resin composite constituting the inner plug 10 may contain a rubber, an elastomer, or the like as other resin components. Examples of the rubber or the elastomer include a natural rubber, a synthetic rubber, and a thermoplastic elastomer such as a styrene-based elastomer or an olefinbased elastomer. Resin components constituting the inner plug 10 may be only a cyclic olefin copolymer and a polyethylene resin or may be a mixture of a cyclic olefin copolymer, a polyethylene resin, other resins, and the like. Examples of other resins which may further be mixed into the foregoing resin composite include a polyolefin resin such as a polypropylene resin.

A proportion of resin components other than a cyclic olefin copolymer and a polyethylene resin (a rubber, an elastomer, a polypropylene resin, and the like) in the foregoing resin composite in 100 parts by weight of the foregoing resin composite is preferably equal to or lower than 20 parts by weight, is more preferably equal to or lower than 15 parts by weight, is further preferably equal to or lower than 10 parts by weight, and is particularly preferably equal to or lower than 5 parts by weight.

Regarding production of the inner plug 10, it is preferable to adopt a method of integrally molding the foregoing resin composite by injection molding or the like. The foregoing resin composite can be prepared by mixing a cyclic olefin copolymer and a polyethylene resin and performing blending through kneading. As described above, other resin components can be added to the foregoing resin composite. Furthermore, within a range not impairing the effects of the foregoing resin composite, in accordance with a desired purpose, an additive agent other than a resin, for example, various kinds of inorganic substances, low molecular organic compounds, or the like can be mixed thereinto. The entire inner plug 10 may be colorless and transparent, and a part or the entirety thereof in a thickness direction or a surface direction may be colored. When the inner plug 10 is transparent, in order to suppress an influence of a beam of light on the content, it is preferable to add a UV absorber or the like.

The inner plug 10 of the embodiment has the nozzle hole 12 penetrating the inner plug 10 in the nozzle part 11. Tips of the nozzle hole 12 and the nozzle part 11 in the eye drop container are subjected to machining with appropriate accuracy for stable dripping of eye drops. A diameter of the nozzle hole 12 is suitably set based on a volume or the like of droplets of eye drops, and examples thereof include approximately 1 to 3 mm. Examples of a length of the nozzle hole 12 include approximately 1 to 10 mm.

Examples of molding the nozzle hole 12 include a method in which a pin-shaped member is input to a mold for injection molding, causing a molten resin to be solidified therearound, forming the nozzle part 11, and then withdrawing the pin-shaped member from the nozzle part 11. In addition, the nozzle hole 12 may be used for causing the nozzle part 11 to hold a cap C, which will be described. For this reason, it is preferable that the inner plug 10 have durability such that cracking due to deformation in the nozzle hole 12 does not occur even if stress is applied to the nozzle hole 12.

The container body 1 has the opening part 2 in which the inner plug 10 is mounted, and a body part 4 which surrounds the storage part 5. A shoulder part 3 which decreases in diameter in stages may be provided between the opening part 2 and the body part 4. It is preferable that at least the container body 1 have a layer including a cyclic olefin copolymer on at least a surface in contact with a liquid content. A resin including a cyclic olefin copolymer used in the container body 1 may be suitably designed from alternatives similar to the resin including a cyclic olefin copolymer used in the inner plug 10 described above. However, these resins may be the same or may be different resins.

The container body 1 may have a reinforcement layer, a gas barrier layer, a UV absorption layer, an oxygen absorption layer, a print layer, or the like. Examples of a method for laminating layers constituting the container body 1 include dry lamination, extrusion lamination, coextrusion, and coating. The laminating method can be suitably selected in accordance with the material, the combination, or the like of layers. The entire container body 1 may be colorless and transparent, and a part or the entirety thereof in a thickness direction or a surface direction may be colored. A method for molding the container body 1 is not particularly limited, and examples thereof include blow molding. A kind of the container of the container body 1 is not particularly limited, and examples thereof include a bottle container.

The eye drop container of the embodiment may have the cap C in order to protect the nozzle part 11. It is preferable that the cap C be coupled to at least any of the nozzle part 11 of the inner plug 10, and the opening part 2, the shoulder part 3, and the body part 4 of the container body 1 in an opening/closing manner or an attachable/detachable manner. A capacity of the storage part 5 is not particularly limited. However, it is 20 ml or smaller, and examples thereof include 3 ml, 5 ml, 10 ml, 15 ml, and 20 ml.

Examples of eye drops filling the eye drop container of the embodiment include aqueous eye drops, oil-based eye drops, use-time soluble eye drops, and suspension eye drops. Eye drops may contain a solubilizer, a stabilizer, an isotonic agent, a buffer agent, a pH regulator, a preservative, a thickening agent, or the like as an additive agent other than active components.

Specific examples of active components of the eye drops filling the eye drop container of the embodiment include isopropyl unoprostone, latanoprost, travoprost, tafluprost, and bimatoprost as prostaglandin-related drugs; diclofenac sodium, pranoprofen, bromphenac sodium hydrate, and nepafenac as non-steroidal anti-inflammatory drugs; cyanocobalamin and flavin adenine dinucleotide sodium as vitamin B preparations; acitazanolast hydrate, amlexanox, ibudilast, epinastine hydrochloride, olopatadine hydrochloride, sodium cromoglycate, ketotifen fumarate, tranilast, pemirolast potassium, and levocabastine hydrochloride as anti-allergic components; cyclosporine and tacrolimus hydrate as immunosuppressive drugs; carteolol hydrochloride, timolol maleate, niprazirol, betaxolol hydrochloride, and levobnorol hydrochloride as β-blockers; bunazosin hydrochloride as an α1-blocker; brimonidine tartrate as an α2-stimulant; pilocarpine hydrochloride as a parasympathomimetic agent; dipivefrine hydrochloride as a sympathomimetic drug; distigmine bromide as a cholinesterase inhibitor; glutathione and pyrenoxin as cataract remedies; gatifloxacin hydrate, dibekacin sulfate, tosufloxacin tosylate hydrate, tobramycin, vancomycin hydrochloride, moxifloxacin hydrochloride, levofloxacin hydrate, lomefloxacin hydrochloride, ofloxacin, chloramphenicol, and norfloxacin as antibacterial drugs; and dorzolamide hydrochloride and brinzolamide as βblockers/carbonic anhydrase inhibitor compounding agents.

Hereinabove, description has been given on the basis of the preferred embodiment of the present invention. However, the present invention is not limited to the embodiment described above, and various modifications and changes can be made within a range not departing from the gist of the present invention.

A dripping container having the inner plug of the embodiment is not limited to an eye drop container and can also be applied to a container for storing other contents. Examples thereof include containers for nasal drops, eardrops, and a chemical agent, a lotion, and the like of a type of dripping a drug solution on a lesion at the time of administration.

Regarding a resin composite including a cyclic olefin copolymer, in place of a cyclic olefin copolymer or together with a cyclic olefin copolymer, a homopolymer of a cyclic olefin or hydrogenated products thereof can be mixed thereinto.

### EXAMPLE

Hereinafter, the present invention will be specifically described using Examples.

### Manufacturing of Inner Plug

Inner plugs having a nozzle were manufactured using resin composites of compositions shown in Tables 1 and 2. A copolymer of cyclic olefins different from each other (COP) and a polyethylene resin (PE) were used for producing the resin composites.

### Measurement of Residual Rate

In each of brown glass bottles having a diameter of 24 mm and a height of 45 mm, one inner plug and 3 ml of a lotion “REVITAL (registered trademark) lotion EX I” (liquid content) of Shiseido Co., Ltd. were input, and the brown glass bottles were sealed with an aluminum tape. The entire inner plugs were immersed into the liquid contents, mouths of the brown glass bottles were sealed using an aluminum tape in lids, and the lotions were stored under an environment of a temperature of 40° C. and a relative humidity of 75%. The concentrations of tocopherol acetate (vitamin E) in the liquid contents before immersion of the inner plugs (initial concentrations) were approximately 477 ppm. The concentrations of the tocopherol acetate (vitamin E) in the liquid contents after being stored for a predetermined period of time (concentrations after immersion) were measured. Based on the initial concentrations and the concentrations after immersion, a residual rate is calculated as in the following Expression 1.

$$\begin{matrix}\text{Residual rate} \left(\%\right) = \\ \left(\text{concentrations after immersion/initial concentrations}\right) = 100 \left(\%\right)\end{matrix}$$

TABLE 1

| No | Composition [parts by weight] | | Residual rate [%] | | | |
|---|---|---|---|---|---|---|
| | COP | PE | Initial | 28 days | 60 days | 90 days |
| 1-1 | 100 | 0 | 100.0 | 95.8 | 93.0 | 94.2 |
| 1-2 | 90 | 10 | 100.0 | 93.6 | 91.8 | 92.2 |
| 1-3 | 80 | 20 | 100.0 | 93.6 | 89.4 | 89.2 |
| 1-4 | 60 | 40 | 100.0 | 90.0 | 82.6 | 81.9 |
| 1-5 | 40 | 60 | 100.0 | 83.8 | 71.0 | 68.6 |
| 1-6 | 20 | 80 | 100.0 | 76.2 | 50.3 | 42.9 |
| 1-7 | 0 | 100 | 100.0 | 71.5 | 39.8 | 32.6 |

Table 1 shows measurement results of the residual rate (%). The residual rate (%) was measured three times after the elapse of 28 days, after the elapse of 60 days, and after the elapse of 90 days while having the proportion corresponding to an initial concentration as 100%. In the inner plugs of the numbers 1-1 to 1-5 molded using resin composites in which COP was blended therein by 40 weight% or higher, the residual rate of the components was high. In contrast, in the inner plugs of the numbers 1-6 and 1-7 molded using resin composites in which the proportion of COP was 20 weight% or lower, the residual rate of the components was extremely low.

### Test of Cracking Stress Durability

A tip of a tool was input to the nozzle parts of the inner plugs by the hand of a worker. Thereafter, the worker thrust it downward by the hand, and stress in the radial direction was added to the nozzle hole via the tip of the foregoing tool. The tip of the foregoing tool had a tapered surface in contact with the inner surface of the nozzle hole so that a downward force could be converted into an outward force in the radial direction. The case in which the inner plug endured stress was evaluated as “O”, and the case in which the inner plug was cracked into two pieces due to stress was evaluated as “X”.

TABLE 2

| No | Composition [parts by weight] | | Cracking stress durability |
|---|---|---|---|
| | COP | LLDPE | |
| 2-1 | 100 | 0 | O |
| 2-2 | 90 | 10 | X |
| 2-3 | 80 | 20 | O |
| 2-4 | 60 | 40 | O |
| 2-5 | 40 | 60 | O |
| 2-6 | 20 | 80 | O |
| 2-7 | 0 | 100 | O |

Table 2 shows test results of cracking stress durability. Linear low-density polyethylene (LLDPE) was used as a polyethylene resin (PE). In the inner plug of the number 2-1 molded from 100% of COP, cracking due to stress did not occur. In the inner plug of the number 2-2 molded using a resin composite in which LLDPE was blended therein by 10 weight%, cracking due to stress occurred. In the inner plugs of 2-3 to 2-7 molded using resin composites in which LLDPE was blended therein by 20 weight% or higher, cracking due to stress did not occur.

### INDUSTRIAL APPLICABILITY

An inner plug of the present invention has a liquid contact part that is constituted of a layer including a cyclic olefin copolymer having excellent non-sorption properties, and a cushioning part that is provided in an outer circumferential part in contact with an opening part. Therefore, sorption (adsorption, absorption) of eye drops can be suppressed. Thus, the present invention can be industrially utilized.

### REFERENCE SIGNS LIST

C Cap
1 Container body
2 Opening part
2a Tip surface of opening part
2b Inner surface of opening part
3 Shoulder part
4 Body part
5 Storage part
10 Inner plug
10a Liquid contact part
10b Inner plug main body
11 Nozzle part
11a Tip of nozzle part
12 Nozzle hole
13 Flange part
14 Leg part
15 Outer circumferential surface of leg part
16 Inner cylinder part
16a Tip of inner cylinder part
17 Clearance part

1. An inner plug to be mounted in an opening part of a container body and constituting a nozzle,
wherein the inner plug is constituted of a resin composite including a cyclic olefin copolymer and a polyethylene resin, and
a proportion of the cyclic olefin copolymer in 100 parts by weight of the resin composite is 25 to 85 parts by weight, a proportion of the polyethylene resin is 15 to 75 parts by weight, and a total amount of the cyclic olefin copolymer and the polyethylene resin is 80 to 100 parts by weight.

2. The inner plug according to claim 1,
wherein the inner plug has a flange part which comes into contact with a tip surface of the opening part, and a leg part which comes into contact with an inner surface of the opening part.

3. The inner plug according to claim 1, wherein the nozzle has a nozzle hole penetrating the inner plug.

4. The inner plug according to claim 1, wherein the resin composite contains a rubber or an elastomer.

5. The inner plug according to claim 1, wherein the resin composite includes at least one of a copolymer of cyclic olefins different from each other (COP) and a copolymer of a cyclic olefin and an acyclic olefin (COC).

6. An eye drop container comprising:
the inner plug according to claim 1; and
a container body that has an opening part in which the inner plug is mounted.

7. The eye drop container according to claim 6,
wherein the container body has a layer including a cyclic olefin copolymer on at least a surface in contact with a liquid content.
